(19) United States
(12) Patent Application Publication
(10) Pub. No.: US 2026/0061019 A1
(43) Pub. Date: Mar. 5, 2026

(54) Topical Compositions For Regulating Sebum Production

(71) Applicant: ELC Management LLC, Melville, NY (US)

(72) Inventors: Jian Cao, Shanghai (CN); Qianwen Fan, Shanghai (CN); Huanjun Zhou, Shanghai (CN); Klodjan Stafa, Ardsley, NY (US); Kelsey Morris, Freeport, NY (US); Thomas Mammone, Farmingdale, NY (US); Nadine Pernodet, Dix Hills, NY (US)

(21) Appl. No.: 19/311,771

(22) Filed: Aug. 27, 2025

Related U.S. Application Data
(60) Provisional application No. 63/688,759, filed on Aug. 29, 2024.

(51) Int. Cl. A61K 36/889; A61K 9/00; A61K 31/13; A61K 36/02; A61P 17/08

(52) U.S. Cl. CPC A61K 36/889; A61K 9/0014; A61K 31/13; A61K 36/02; A61P 17/08

(57) ABSTRACT

Compositions comprising Serenoa serrulata (saw palmetto) fruit extract, Laminaria saccharina (brown algae) extract, and Glycyrrhiza inflata root extract are effective inhibit the activity of 5-alpha Reductase; lower neutral lipid content; and reduce expression of SREBP1, FASN and ACACA, in human sebocytes. By regulating excess sebum production, compositions of the invention are useful to improve the health and appearance of skin.

### FIELD OF THE INVENTION

The present invention is in the field of topical skincare formulations. Specifically, it pertains to topical compositions that regulate the production of sebum in human skin.

### BACKGROUND OF THE INVENTION

Sebaceous glands are located in the skin, over most of the body. A main purpose of sebaceous glands is to produce and secrete sebum. Human sebum is a rich mixture of lipids: tri- and di-glycerides and free fatty acids (50-60%), wax esters (20-30%), squalene (10-16%), and cholesterol (2-4%). Sebum is a necessary component of healthy skin. Beneficial functions of sebum include photoprotective, anti-microbial and pro- and anti-inflammatory effects. The main cells of sebaceous glands are sebocytes. Sebocytes are terminally differentiated epithelial cells that produce and store sebum. Eventually, fully mature sebocytes disintegrate (a process known as holocrine secretion) which releases the stored sebum into the canal of an associated hair follicle or into a duct that opens directly onto the surface of the skin. The lifespan of sebocytes is approximately 21 to 25 days. Due the their continual destruction, sebocytes are continually replaced by mitosis. Their proliferation and development is largely controlled by androgen hormones.

As noted, sebum is a necessary component of healthy skin. Nevertheless, excessive secretion of sebum leads to problems, such as shiny, greasy and acned skin. Sebum secretion is affected by a variety of factors, including genetics, psychological stress, diet, humidity, and hormone production. It is now understood that the human skin is steroidogenic; that is, capable of producing glucocorticoids, androgens and estrogens. Overproduction of androgen hormones, such as testosterone and DHT, leads to excessive production of sebum, which is especially true in men. In the sebaceous glands, cholesterol undergoes a series of enzymatic steps to be converted into testosterone and DHT. First, cutaneous cholesterol is converted into pregnenolone, which, in turn, is converted into the hormone dehydroepiandrosterone (DHEA). DHEA is subsequently converted (by 3β-hydroxysteroid dehydrogenase) into androstenedione. Then, (via the action of 17β-hydroxysteroid dehydrogenase) androstenedione is converted into testosterone. Testosterone may be reduced into dihydrotestosterone (DHT, the most potent androgen) by the action of 5α-Reductase, with the participation of NADPH. However, DHT may also be produced when 5α-Reductase converts androstenedione into 5α-androstanedione, which is subsequently converted to DHT (by 17βHSD). In the cytoplasm, both testosterone and DHT bind to androgen receptors, which subsequently relocate into the cell nucleus, bind to specific nucleotide sequences of the chromosomal DNA, and influence transcriptional activity of certain genes. In this way, testosterone and DHT strongly modulate sebocyte differentiation, sebum production, and inflammatory cascades.

SREBP1 (sterol regulatory element-binding protein-1) is a transcription factor that plays a central role in lipogenesis, including controlling the expression of FASN and ACACA. FASN and ACACA are genes that encode fatty acid synthase (FASN) and acetyl-CoA carboxylase (ACACA), respectively, enzymes involved in fatty acid synthesis. Thus, down-regulation of the gene that encodes SREBP1 leads to reduced production of FASN and ACACA enzymes, and a reduced rate of lipogenesis.

The foregoing suggests that the regulation of sebum production may be achieved through one or more interventions, including: inhibition of 5α-Reductase (also known as 3-oxo-5α-steroid 4-dehydrogenases) to inhibit the over-production of testosterone and DHT; modulating the level of neutral lipids (such as wax ester) in sebocytes; and reducing the expression of genes that are significant in lipogenesis. While the use of 5α-Reductase inhibitors for preventing excess sebum production is known in the cosmetic and dermatological realms, a single, stable, commercially viable composition that is effective to inhibit the activity of 5-alpha Reductase, and lower neutral lipid content, and reduce expression of SREBP1, FASN and ACACA is unknown.

### SUMMARY OF THE INVENTION

Compositions of the invention include combinations of Serenoa serrulata (saw palmetto) fruit extract, Laminaria saccharina (brown algae) extract, and licochalcone A (C₂₁H₂₂O₄). In primary sebocytes, compositions of the invention are effective to 1) inhibit the activity of 5-alpha Reductase; 2) lower neutral lipid content; 3) reduce expression of SREBP1, FASN and ACACA. By regulating excess sebum production, the present invention improves the health and appearance of skin.

### DESCRIPTIONS OF THE FIGURES

FIG. 1 is a graph that shows the ability of Serenoa serrulata fruit extract, Laminaria saccharina extract, licochalcone A, and combinations thereof, to modulate neutral lipid content in SZ95 immortalized sebocytes.

FIGS. 2-4 show the ability of Laminaria saccharina extract and licochalcone A, and combinations thereof, to decrease the production of SREBP1, FASN and ACACA in mature sebocytes.

### DETAILED DESCRIPTION OF THE INVENTION

The dimensions and values disclosed herein are not to be understood as being strictly limited to the exact numerical values recited. Instead, unless otherwise specified, each such dimension is intended to mean both the recited value and a functionally equivalent range surrounding that value. Except where otherwise indicated, all numbers in this description indicating amounts or ratios of material or conditions of reaction, physical properties of materials and/or use are to be understood as modified by the word “about”. Unless otherwise specified, all percentages refer to the total weight of the final composition.

As used herein, the term “comprise” means that a collection of elements is not necessarily limited to those explicitly recited.

5α-Reductase is expressed in various tissues, including the liver. We tested various blends of three active ingredients, Serenoa serrulata (saw palmetto) fruit extract, Laminaria saccharina extract, licochalcone A for their capacity to inhibit 5α-Reductase enzymatic activity in human liver microbiomes (HLM). These three materials were also tested for their ability to modulate neutral lipid content in sebocytes, and for their ability to lower production of SREBP1, FASN and ACACA in sebocytes. Results show that combinations of these three ingredients at certain concentrations are effective to do all three; inhibit 5α-Reductase enzymatic activity, modulate lipid content and decrease lipogenesis. This was unexpected.

The following tests were conducted to demonstrate the unexpected effectiveness of the combination of Serenoa serrulata (saw palmetto) fruit extract, Laminaria saccharina extract, and licochalcone A to simultaneously inhibit the activity of 5-alpha Reductase, lower neutral lipid content, and reduce expression of SREBP1, FASN and ACACA in cells.

### Active Materials for Testing

Serenoa serrulata fruit extract (saw palmetto fruit extract) is commercially available, for example, as ViaPure® Sabal from Actives International. ViaPure® Sabal is, on average, 100% Serenoa serrulata fruit extract. Laminaria saccharina extract is available from Seppic as Phlorogine™ BG/PF (INCI name: water (and) butylene glycol (and) Laminaria saccharina extract). The material Phlorogine™ BG/PF material used in the testing described below comprises 1.51% Laminaria saccharina extract. Licochalcone A may be extracted from Glycyrrhiza inflata root. Licochalcone A is commercially available, for example, as Licochalcone A 21% from Taos, Inc (Fairfield, NJU). Licochalcone A 21% is 21% licochalcone A.

### I. Inhibitory Effects of Actives on 5α-Reductase Activities

The inhibitory effect of certain actives on 5α-Reductase was determined by measuring the change in testosterone content in a reaction system containing the enzyme. Serenoa serrulata (saw palmetto) fruit extract, Laminaria saccharina (brown algae) extract, and licochalcone A were tested at various levels, individually and in combination.

### Enzyme Reaction System

Seven test samples were prepared, as shown in Table 1. An eighth test sample, Ethanol (not shown in Table 1) was used as a negative control as shown in Table 3.

TABLE 1

| | Test | Test | | | | | |
|---|---|---|---|---|---|---|---|
| Reagent | Sample 1 | sample 2 | | | | Test | |
| (starting | (blank | (quality | | Test | | Samples | |
| concentration) | control) | control) | | Sample 3 | | 4-7 | |
| Tris-HCl buffer, pH 7.4 | 70 μL | 65 | μL | 65 | μL | 65 | μL |
| HLM (10 mg/mL) | — | 5 | μL | 5 | μL | 5 | μL |
| Active Tested | — | — | | — | | 10 | μL |
| Dutasteride (0.5 | — | — | | 10 | μL | — | |
| mg/mL) | | | | | | | |
| Vehicle (Tris-HCl | 10 μL | 10 | μL | — | | — | |
| buffer) | | | | | | | |
| Testosterone (4 μg/mL) | 10 μL | 10 | μL | 10 | μL | 10 | μL |
| NADPH (10 mmol/L) | 10 μL | 10 | μL | 10 | μL | 10 | μL |

In Table 1, HLM refers to human liver microsomes, a source of 5α-Reductase (XenoTech H0610, Kansas City, KS). Test sample 1 is a blank control. Test sample 2 is a response group without inhibitor actives. Test sample 3 is a positive control (Dutasteride is a known inhibitor of 5α-Reductase activity). Test samples 4-6 contain one of the active ingredients (Serenoa serrulata (saw palmetto) fruit extract, Laminaria saccharina extract, licochalcone A). Test sample 7 contains a combination of all three actives. Q designates no active in the sample; R indicates the presence of one or more actives in the sample. Trials for Test samples 4-8 were repeated at different levels of active ingredients, as shown in Table 2.

TABLE 2
Concentration of Actives Tested (test samples 4-8)

| Test | | |
|---|---|---|
| Sample | Active tested | % levels tested |
| 4 | ViaPure ® Sabal (100% | 0.025, 0.05, 0.1, 0.2, 0.4 |
| | Serenoa serrulata fruit ext. | |
| 5 | Phlorogine ™ BG/PF | 0.125, 0.25, 0.5, 1, 2 |
| | (1.51% Laminaria | |
| | saccharina extract) | |
| 6 | Licochalcone A 21% (21% | 0.00125, 0.0025, 0.005, 0.01, 0.02 |
| | licochalcone A) | |
| 7 | ViaPure ® Sabal | 0.1 |
| | Phlorogine ™ BG/PF | 0.5 |
| | Licochalcone A 21% | 0.005 |
| 8 | Ethanol (negative control) | 0.625, 1.25, 2.5 |

The total volume of each reaction system was 100 μl. Reaction temperature was 37° C. After 30 minutes, 50 μL of reaction liquid was added to 100 μL methanol solution to terminate the reaction. The mixture was centrifuged (10000×g for 10 minutes) to collect the supernatant. Quantitative determination of the amount of testosterone in the supernatant of each Test Sample 1-8 was determined via ultra-high performance liquid phase tandem mass spectrometry system (UPLC-MS).

The specific UPLC-MS system employed included the Q Exactive quadrupole/electrostatic field orbital trap high resolution mass spectrometer (Thermo Fisher©, USA) equipped with an H-ESI II ion source. The liquid chromatography system is Dionex Ultimate 3000 high pressure liquid chromatography. The chromatography was performed on Waters Acquity HSS T3 column (100×2.1 mm, 1.8 μm) with mobile phases consisting of 0.1% formic acid water (A) and acetonitrile (B). Gradient elution was performed for 0-0.5 min, 45% B; 0.5-2.0 min, 45%-55% B; 2.0-3.2 min, 55-60% B; 3.2-3.5 min, 60-90% B; 3.5-5.0 min, 90% B; 5.0-6.0 min, 45% B; flow rate: 0.4 mL/min; sample size: 5 μL; column temperature: 40° C. The mass spectrum was analyzed by electrospray ion source positive ion mode, and the data was collected by full scan-SIM mode, with a range of 100˜1500 m/z and a resolution of 70,000. The spray voltage was 3.5 kV. Capillary temperature was 320° C. Heater temperature was 300 C. Sheath gas flow was 30 Arb. The auxiliary gas flow rate was 13 Arb. Automatic gain control (AGC) was 106. A testosterone certified standard at five concentrations ranging from 12.5 to 200 ng/ml in solution, was used to calibrate the UPLC-MS technique.

### Data Analysis

From the chromatograph elution curves, the area under each detection peak was measured, and plotted against the level of testosterone detected in the standard sample. Using regression analysis, the following linear equation was obtained to relate detection peak area to testosterone content:

$$Y = 918382.1 \; 206 X + 3149551.9093 ( {R}^{2} = 0.9 \; 966 ) , \qquad ( Equation 1 )$$

where the X coordinate represents testosterone concentration, and the Y coordinate represents the detection peak area.

Thereafter, each Test Sample 1-8 was analyzed by UPLC-MS, and the testosterone content was determined from equation 1. Next, the percent change in testosterone content due to the active ingredient was calculated as follows:

$$I ( \% ) = [ ( B - Q ) - ( B - R ) ] / ( B - Q ) \times 100 , \qquad ( Equation 2 )$$

where B represents the testosterone content of the blank control group, Q represents the testosterone content of the quality control group (response group without inhibitor), and R represents the testosterone content of the response group with one or more actives being tested for inhibition. Any change in testosterone content is due to consumption of testosterone by 5α-Reductase enzyme activity, and reflects the strength of 5α-Reductase enzyme activity. For example, when the amount of testosterone consumed by 5α-Reductase in the Test Samples with active ingredient is lower than the amount of testosterone consumed in the Test Samples without active ingredient, then the active ingredient is considered to inhibit 5α-Reductase. Alternatively, when the testosterone content of the Test Samples with active ingredient is greater than the testosterone content of the Test Samples without active ingredient, then the active ingredient is considered to inhibit 5α-Reductase. Statistical analysis was conducted by t-test method, compared with the quality control group.

### Results

Table 3 shows the inhibitory effect of each tested active on 5α-Reductase. The results indicate that all three of the tested actives are individually effective to inhibit the enzymatic conversion of testosterone by 5α-Reductase. For example, 0.1% ViaPure® Sabal (0.1% Serenoa serrulata fruit extract), 0.5% Phlorogine™ BG/PF (0.00755% Laminaria saccharina extract), and 0.005% Licochalcon A 21% (0.00105% licochalcone A) significantly inhibited 5α-Reductase activities by 88.79%, 45.99% and 94.31%, respectively. Also, at the concentrations tested, Serenoa serrulata fruit extract and licochalcone A are comparable or superior to the Dutasteride control, while Laminaria saccharina extract is significantly less effective than Dutasteride. The combination (0.1% Serenoa serrulata fruit extract, 0.00755% Laminaria saccharina extract, and 0.00105% licochalcone A exhibited a remarkable inhibitory effect of 99.87%. The effectiveness of all three actives is uniformly dose-dependent, so that the demonstrated efficacy is expected to be valid for all concentrations in the tested ranges (0.025-0.4% by weight of Serenoa serrulata fruit extract; 0.001888-0.0302% by weight of Laminaria saccharina extract; 0.000263-0.0042% by weight of licochalcone A), and slightly wider; (0.02-0.5% by weight of Serenoa serrulata fruit extract; 0.001888-0.0302% by weight of Laminaria saccharina extract; 0.000263-0.0042% by weight of licochalcone A). By comparison, typically recommended levels of licochalcone A are 0.01-0.05% by weight, which is at least ten times greater than in the present invention.

TABLE 3

| | | 5α reductases |
|---|---|---|
| | | inhibitory effect |
| Test Sample | Active fraction | (average %) |
| Quality Control NT | | 0.00 |
| Dutasteride (0.05 mg/mL) | | 86.09 |
| 0.40% ViaPure ® Sabal | 0.40% | 109.99 |
| 0.20% ViaPure ® Sabal | 0.20% | 94.14 |
| 0.10% ViaPure ® Sabal | 0.10% | 88.79 |
| 0.05% ViaPure ® Sabal | 0.05% | 80.11 |
| 0.025% ViaPure ® Sabal | 0.025% | 71.39 |
| 2.00% Phlorogine ™ BG/PF | 0.0302% | 70.73 |
| 1.00% Phlorogine ™ BG/PF | 0.0151% | 60.84 |
| 0.50% Phlorogine ™ BG/PF | 0.00755% | 45.99 |
| 0.25% Phlorogine ™ BG/PF | 0.003775% | 6.78 |
| 0.125% Phlorogine ™ BG/PF | 0.001888% | 6.40 |
| 0.020% Licochalcone A 21% | 0.0042% | 98.03 |
| 0.010% Licochalcone A 21% | 0.0021% | 96.12 |
| 0.0050% Licochalcone A 21% | 0.00105% | 94.31 |
| 0.0025% Licochalcone A 21% | 0.000525% | 71.49 |
| 0.00125% Licochalcone A 21% | 0.000263% | 39.20 |
| ViaPure ®: 0.1%, Phlorogine ™: | 0.1%, 0.00755%, | 99.87 |
| 0.5%, Licochalcone A 21%: | 0.00105% | |
| 0.005% | | |
| 5.0% Ethanol | 5.00% | 0.36 |
| 2.50% Ethanol | 2.50% | −1.82 |
| 1.25% Ethanol | 1.25% | 6.29 |
| 0.625% Ethanol | 0.625% | −3.79 |

### II. Ability of Actives to Modulate Neutral Lipid Content in Sebocytes

The ability of Serenoa serrulata (saw palmetto) fruit extract, Laminaria saccharina extract, and licochalcone A to regulate the levels of neutral lipids in immortalized sebocytes is demonstrated by immunofluorescence.

Human immortalized SZ95 sebocytes originating from human facial sebaceous glands, were cultured in Sebomed® Basal Medium (Sigma-Aldrich) supplemented with 10% fetal bovine serum (Corning), 1 mM CaCl2, 5 ng/ml human recombinant epidermal growth factor (Pufei, China), 1% Anti-Anti (Gibco) at 37° C. in a 5% CO₂ humidified incubator. The medium was changed every other day, and cells were sub-cultured at 60-80% confluence. Then the cells were seeded onto Perkin-Elmer ViewPlate-96 black plates with glass bottom, at 10,000 cells/well. For each plate, only the center 60 wells were used. Wells were treated with one or more actives, at various concentrations. Isotretinoin (25 μM) was used as a positive control.

After incubation, the cells were washed twice with DPBS buffer, and fixed in 4% paraformaldehyde for 5 minutes. Then, 100 μl of a 1 μg/ml Nile red (Sigma-Aldrich) solution in phosphate-buffered saline was added to each well, and incubated at 37° C. for 20 minutes. Fluorescence was measured on an Operetta CLS high content imaging system. Results were expressed as the relative fluorescence units per cell using 485 nm excitation and 565 nm emission wavelengths for neutral lipids, and 540 nm excitation and 620 nm emission wavelength for polar lipids. Nuclear DNA was labelled with DAPI (4′,6-diamidino-2-phenylindole). The lipid accumulation value was calculated using the following equation:

$$\% lipid accmulation = ( {MFI}_{active} / {MFI}_{control} ) \times 100 \% ,$$

where MFI stands for mean fluorescent intensity. Cell viability after sample treatment may be calculated as follows:

$$\% viability = {( DAPI counts )}_{active} / {( DAPI counts )}_{control} \times 100 \%$$

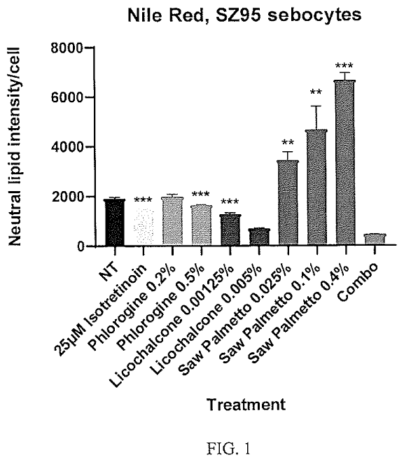

The quantification of neutral lipid content of SZ95 immortalized sebocytes is shown in FIG. 1. Data are represented as means±standard deviation (** means p<0.01; *** means p<0.001; **** means p<0.0001). In immortalized sebocytes, when compared to the negative control (NT) and the positive control, Laminaria saccharina extract (Phlorogine™ BG/PF), by itself, was not effective to alter the neutral lipid content in sebocytes. Licochalcone A, by itself, was somewhat effective to reduce the neutral lipid content. Serenoa serrulata fruit extract, by itself, increased neutral lipid content, substantially. However, and surprisingly, the combination of all three actives (0.1% Serenoa serrulata fruit extract, 0.5% Phlorogine™ BG/PF (0.00755% Laminaria saccharina extract), 0.005% Licochalcone A 21% (0.00105% licochalcone A) performed the best, decreasing neutral lipid content to levels well below the positive control or the individual actives.

III. Ability of Actives to Reduce Expression of Genes that Encode SREBP1 in Mature (68 Year Old) Sebocytes.

The effect of Laminaria saccharina extract, licochalcone A and a combination of the two on the expression of SREBP1 in mature (68 years old) sebocytes was studied with qPCR. Test samples were prepared according to Table 4.

TABLE 4

| Reagent | Test Sample Preparation |
|---|---|
| media (0.0125% ethanol) | 1.75 μl of 100% ethanol in 13.9982 of media |
| tretinoin 5 μM | 2.8 μl of 25 mM in 13.9972 ml of media |
| Licochalcone A 21% | 1.75 μl of 1% extract in 13.9982 ml of media |
| (0.000125%) | |
| Phlorogine ™ BG/PF | 70 μl of 100% extract to 13.93 ml of media |
| (0.5%) | |
| Licochalcone A 21% | 1.75 ul of 1% Lico in 6.9982 ml of media + |
| (0.000125%) and | 70 μl of 100% Phlorogine ™ BG/PF in |
| Phlorogine ™ BG/PF | 6.930 ml of media |
| (0.5%) | |
| Fatostatin 5 μM | 2.667 μl of 30 mM stock to 15.997 ml of media |

Tretinoin (retinoic acid) and fatostatin are positive controls. Fatostatin is a diarylthiazole derivative that impairs the activation process of SREBPs, thereby decreasing the transcription of lipogenic genes in cells. We plated 42 wells with 150 k sebocytes per well, and allowed the cells to adhere overnight at 37 C, 5% CO₂. Cells were given 2 ml of test sample preparation per well, and let stand for 72 hours. Thereafter, RNA was extracted from the cells by scraping with lysis buffer, and processing the cell lysate via the QICube (Qiagen). The RNeasy mini kit by Qiagen was used. Quantification of RNA was determined using the Quant-it RiboGreen RNA Assay Kit by ThermoFisher©. From each sample, 30 ng of RNA was used to create cDNA using SuperScript IV Vilo Master Mix by ThermoFisher©.

cDNA samples were assayed with kits appropriate for detecting SREBP1, FASN and ACACA, as well as GAPDH, which is commonly used as an endogenous control that allows relative gene expression quantification in cDNA samples. All probes were sourced from ThermoFisher© Scientific.

A PCR reaction mix was prepared for each cDNA sample according to Table 5.

TABLE 5

| | |
|---|---|
| Reaction Component | μL/Reaction |

| | | |
|---|---|---|
| TaqMan Fast Universal PCR Master Mix (2X) | 10 | μL |
| 20X TaqMan Gene Expression Assay Mix | 1 | μL |
| cDNA sample | 1 | μL |
| Nuclease-free H₂O | 8 | μL |
| Total | 20 | μL |

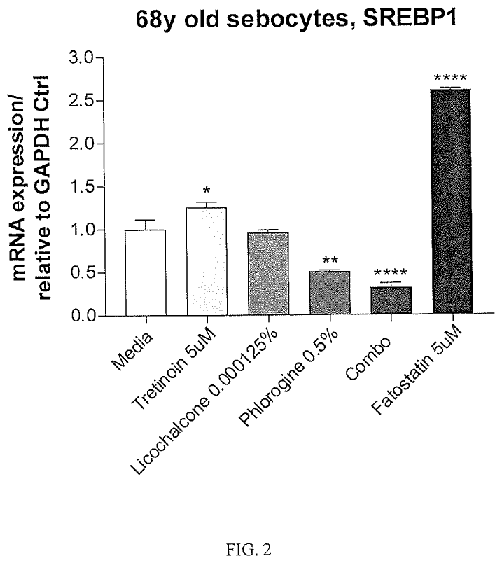

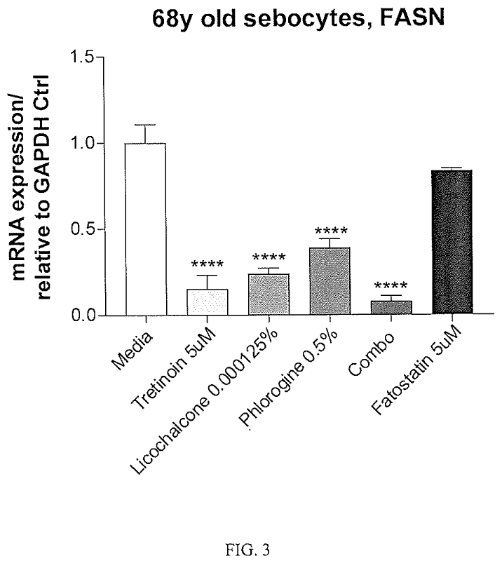

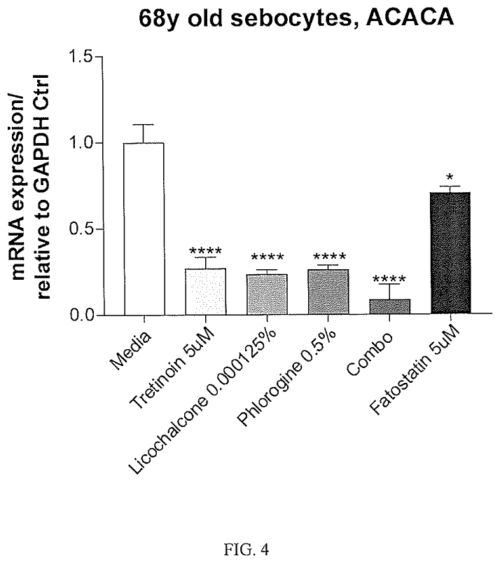

Solutions were prepared for plating by adding 76 μL of the PCR Reaction Mix to 4 μL of cDNA sample; cap, vortex and quick spin. For each cDNA solution, a 96 well plate was prepared by loading 20 μL of solution, per well. The plates were sealed with an optical adhesive cover, inverted to mix, and centrifuged at 1000 rpm for approximately thirty seconds. Quantification of mRNA was made using the Quant Studio 7 Flex qPCR machine. We cycled each sample (55 cycles) as follows: heating stage: 95° C. for twenty seconds; PCR stage: 95° C. for two seconds; anneal at 60° C. for twenty seconds; hold stage 4° C. for 320 seconds. The results are graphed in FIGS. 2-4 (* means p<0.05; ** means p<0.01; *** means p<0.001; **** k means p<0.0001). The graphs show the ability of Laminaria saccharina extract (Phlorogine™ BG/PF), licochalcone A, and combinations thereof, to reduce the production of SREBP1, FASN and ACACA in mature sebocytes. Laminaria saccharina extract and licochalcone A, alone and in combination are effective to decrease the gene expression levels of lipogenic targets SREBP1, FASN & ACACA in sebocytes. The combination is especially effective, reducing the expression of SREBP1, FASN & ACACA in sebocytes even more than tretinoin and fatostatin.

We have shown unexpected effectiveness of the combination of Serenoa serrulata (saw palmetto) fruit extract, Laminaria saccharina extract, and licochalcone A to simultaneously inhibit the activity of 5-alpha Reductase, lower neutral lipid content, and reduce expression of SREBP1, FASN and ACACA. It was unknown that a composition with all three actives could simultaneously exploit all three methods of regulating excess sebum production in sebocytes. The practical use of these insights is in products for topical application to the skin. The term “topical application”, as used herein, means to apply or spread the compositions of the present invention onto the surface of the skin. The present invention includes topical compositions that are cosmetically and dermatologically acceptable, and useful for regulating the production of sebum in human skin. The term “dermatologically-acceptable,” as used herein, means that the compositions or components thereof so described are suitable for use in contact with human skin without undue toxicity, incompatibility, instability, allergic response, and the like.

### IV. Topical Compositions

Topical compositions of the invention are generally aqueous, comprising 40%-85% water by weight of the topical composition, for example. Forms may include gels, creamy emulsions, watery essences. In addition to sebum control, the compositions may function as cleansers, moisturizers, toners, and mattifiers. In general, compositions of the invention may comprise those inert and active ingredients that are commonly found in topical cosmetic products, provided the ingredients do not interfere with the ability of the combination of Serenoa serrulata fruit extract, Laminaria saccharina extract, and licochalcone A to simultaneously inhibit the activity of 5-alpha Reductase, lower neutral lipid content, and reduce expression of SREBP1, FASN and ACACA. Nonlimiting examples of cosmetically acceptable skin care actives include peptides, farnesol, bisabolol, phytantriol, glycerol, urea, amino acids, ascorbic acid, vitamin A (e.g., retinoid derivatives such as retinyl palmitate or retinyl proprionate), vitamin E (e.g., tocopherol acetate), vitamin B₃ (e.g., niacinamide) and vitamin B₅ (e.g., panthenol); N-acetyl-D-glucosamine; anti-acne medicaments (for example, resorcinol and salicylic acid); sebum reducers (such as zinc PCA); antioxidants; flavonoids; skin soothing and healing agents (such as aloe vera extract, allantoin, lactobacillus ferment, and soybean extract); moisturizers (such as castor oil, hyaluronic acid and algae extract), and combinations thereof. Skin care actives may be included up to 10% by weight of the topical composition.

In general, compositions of the invention may comprise ingredients that impart or enhance particular aesthetic characteristics to the composition. Examples of ingredients that may be included for aesthetic purposes include essential oils, fragrances, thickeners, opacifiers, humectants, aromatic compounds, emulsifiers (such as Soja (soybean) seed extract) and emollients (such as propanediol). Aesthetic ingredients of this type may be included up to 10% by weight of the topical composition.

For chemical and thermodynamic stability, preservatives, chelators and pH adjusters will typically be included in topical compositions according to the present invention. Stabilizers and preservatives may be included up to 5% by weight of the topical composition.

Topical compositions of the invention may function as sunscreen products. To that end, topical compositions of the invention may include up to 40% of sunscreen-active ingredients that are effective to attenuate the effects of UVA light, UVB light or both. In some embodiments of the invention, non-chemical sunscreen agents are preferred, such as titanium dioxide and iron oxide. In some embodiments, avobenzone is a preferred chemical sunscreen agent.

The technology of the invention may be incorporated into specifically skin care products, but also in color cosmetics, such as foundations, concealers, pressed powders, tinted moisturizers, blush, and eyeshadow. To that end, compositions of the invention may comprise 0.01% to 10% of one or more of organic and inorganic pigments, lakes or dyes.

Following is an example of a specific embodiment of topical compositions according to the present invention. The example composition is not intended to be limiting thereof. Other modifications can be undertaken by the skilled artisan without departing from the spirit and scope of this invention.

### EXAMPLE

| | Percent by weight |
|---|---|
| | of composition |
| Zinc PCA | 0.10 |
| ViaPure ® Sabal* | 0.10 |
| Salicylic acid | 0.18 |
| Propanediol | 5.00 |
| Phlorogine ™ BG/PF** | 0.50 |
| N-acetyl-D-glucosamine | 1.00 |
| Emulsifier(s) | 0.50 |
| Licochalcone A 21%*** | 0.005 |
| Lactobacillus ferment | 1.00 |
| Moisturizer(s) | 1.24 |
| Humectant(s) | 2.00 |
| Waxes | 3.00 |
| pH adjuster(s) | 0.50 |
| Preservative(s) | 0.50 |
| Algae extract | 0.50 |
| Fragrance | 0.30 |
| Water | to 100% |

*Serenoa serrulata (saw palmetto) fruit extract (100%)
**water/butylene glycol/laminaria saccharina (1.51%)
***Licochalcone A (21%)

What is claimed is:

1. A topical composition for regulating the production of sebum in human skin comprising Serenoa serrulata fruit extract, Laminaria saccharina extract, and licochalcone A, wherein:
the concentration of Serenoa serrulata fruit extract ranges from 0.02% to 0.5%, by weight of the topical composition
the concentration of Laminaria saccharina ranges from 0.0015% to 0.03%, by weight of the topical composition
the concentration of licochalcone A ranges from 0.00025% to 0.004%, by weight of the topical composition.

2. A topical composition according to claim 1 further comprising 40%-85% water, by weight of the topical composition.

3. A topical composition according to claim 1 further comprising 0.01% to 10% of one or more organic or inorganic pigments, lakes or dyes.

4. A topical composition according to claim 1 further comprising one or more chemical or non-chemical sunscreen agents.
